(19) United States
(12) Patent Application Publication
(10) Pub. No.: US 2005/0113879 A1
(43) Pub. Date: May 26, 2005

(54) Urinary regulation utilizing actual neuro-coded signals

(76) Inventors: Eleanor Schuler, Rio Rancho, NM (US); Claude K. Lee, Reno, NV (US)

Correspondence Address: BARNES & THORNBURG, P.O. BOX 2786, CHICAGO, IL, 60690-2786, US

(21) Appl. No.: 10/982,093

(22) Filed: Nov. 4, 2004

Related U.S. Application Data
(60) Provisional application No. 60/525,480, filed on Nov. 26, 2003.

(51) Int. Cl. A61N 1/18

(52) U.S. Cl. 607/40

(57) ABSTRACT

A method and device for urinary function control. The method comprises selecting neuro-electrical coded signals, which are similar to those naturally generated within the body, from a storage area that are representative of urinary function. The selected neuro-electrical coded signals are then transmitted or conducted to a treatment member, which is in direct contact with the body, and which then broadcasts the neuro-electrical coded signals to a specific urinary nerve to modulate the urinary function. A control module is provided for transmission to the treatment member. The control module contains the neuro-electrical coded signals which are selected and transmitted to the treatment member, and computer storage can be provided for greater storage capacity and manipulation of the neuro-electrical coded signals.

### RELATED APPLICATIONS

This is the non-provisional filing of application Ser. No. 60/525,480, filed on Nov. 26, 2003, entitled “Urinary Bladder System Regulation Utilizing Actual Neuro-Coded-Signals.”

### BACKGROUND OF THE INVENTION

The storage of urinary waste products in the urinary bladder and the discharging of those waste products outside the body is an ongoing process relating to body homeostasis (Life equilibrium). Bodily homeostasis is the regulation of the milieu interieur (internal environment) of the living mammalian body to maintain balance among the various vital organs that are required to maintain healthy life. Urinary bladder homeostasis is the process through which the bladder accepts the liquid urine waste product received from the kidneys. When the bladder reaches a certain fullness it releases (voids) urine to the world outside the body. The process includes, but is not limited to the dumping and removal from the bladder of toxins, invading microbes, environmental dust, metabolic by-product wastes and certain water soluble odors. The bladder accepts most medication metabolic waste products after the liver and/or kidney(s) have processed them.

Urination (micturition) is the process of removing waste liquefied products delivered from the kidney(s) to the urinary bladder via the ureter(s). The bladder accumulates the waste liquid. (called urine). The autonomic nervous system signaling via sympathetic and parasympathetic nerves release the urine out of the body via the urethral and bladder muscles working together.

There are a number of medical conditions which interfere with the normal function of collecting, storing and discharging urine involving the urinary bladder and associated structures. Some of these medical conditions are the common urinary tract disorders of frequent urination; too much urination (polyuria); slow urination (oliguria); no urination (anuria) and voiding during the night (nocturia). Other conditions such as bed-wetting (enuresis); painful urination (dysuria) and the multiple problems of hesitancy, straining and decreased force (obstrtctive symptoms) affect many children and adults. Also there is the uncontrollable loss(dripping or voiding) of urine, known as incontinence, which affects a significant number of people and animals.

In addition there are miscellaneous trauma injuries, infections and cancer invasion which cause malfunction of the urinary bladder system. The above listed and other medical difficulties of the urinary bladder system are treated presently by surgery, medications, exercises and a few electrical stimulation devices.

Myoneurogenic (nerve & muscle) disorders are associated with a number of dysfunctions of the urinary bladder system some of which have had treatments devised and others which cause considerable distress for those persons affected, and have no effective treatments.

Presently there are no treatments available to treat any of the conditions listed herein which utilize the actual neuro-electrical coded signals that normally operate the urinary bladder system. Such neuro-electrical coded signals originate in the brain and brainstem.

The inventors provide a medical treatment method and device which utilize codes similar to the actual body generated neuro-electrical coded signals that normally operate and regulate the urinary bladder system. The new medical treatment invention described herein provides approaches for a range of bladder and urinary system dysfunction, that previously have not been available.

Normal bladder activity depends on both autonomic nerve activity and voluntary sympathetic nerve activity to achieve the purpose of ridding the body of urine. Reflex actions as well as active electrical neuro-electrical coded signals play a role in the complex regulation of bladder duties and activities. The bladder wall consists of smooth muscle fibers that form an interlaced complicated network consisting of three layers. The detrusor muscle fibers are interwoven into the muscularis of the bladder. The distention of the bladder wall caused by accumulating urine activates the detrusor muscle to cause contraction of the bladder until it is empty. Mankind or animals can void with a partially filled bladder and can interrupt the urine evacuation at will, via sympathetic neuro influence.

The urinary bladder collects urine from the kidneys. Kidneys are an excretory gland plus a vital organ. They produce hormones involved in the control of blood pressure and for erythropoiesis which is the production of red blood cells. Additionally, the kidney functions as a vital organ filter to remove soluble waste products from the blood stream. Therefore, the kidney is part a method and function to remove certain liquid waste and it is an endocrine gland too. Kidney(s) drain the waste liquid (urine) by means of the ureter(s) which carry the liquid into the urinary bladder.

Longitudinal fibrous bundles in the anterior wall of the bladder extend into the proximal urethra which ultimately evolves into the urinary sphincter. The internal sphincter is composed of muscle bundles which are looped around the urethral orifice. The muscular orifice retains urine until neuro-electrical coded signals cause urethral strictures to relax and allow urine flow to the outside world. Urine flow, under certain conditions, is initiated without a neuro-electrical coded signal being received by the bladder in a sort of automatic behavior.

Sympathetic nerves which control the bladder and the lower ureter(s) travel from the kidneys and are enervated from lumbar spinal cord L-1 & L-2 which travels through the sacral splanchnic nerves to the hypogastric plexus. While the parasympathetic urination activation nerves arise from the sacral spinal cord in position S-2 & S-4 and are distributed into the bladder wall plexus via the pelvic splanchnic nerves. The sympathetic activation nerves relax the detrusor muscles and contract the internal sphincter. Parasympathetic nerves contract the detrusor muscle and relax the internal sphincter which causes emptying of the urinary bladder.

Essential to urination is the relaxation of the pelvic floor muscles which leads to shortening of the urogenital diaphragm and elevation of the bladder and the prostate (in males). In addition, the urethra, vagina (in females) and rectum are stretched. The levator anal muscle of the annus is also elevated. In the male, striated muscles which surround the proximal urethra are connected to the vesical trigone muscle where it can close the vesical orifice and prevents reflux of semen into the bladder after ejaculation.

The entire process of storing urine and detecting neurologically when it is full enough to void is detected by afferent neuro-stretch receptors in the bladder wall. Innervation of the bladder and urethral control mechanisms contain both facilitatory and inhibitory efferent motor nerves as well as sensory afferent nerves. The thalamus, amygdala, hippocampus and other brain structures play a role in the control of the actions of the urinary bladder. In addition the pons and medulla oblongata play an important role in the micturition process.

### SUMMARY OF THE INVENTION

The invention provides a method for controlling urinary function. Stored neuro-electrical coded signals that are generated and carried in the body are selected from a storage area. The selected waveforms are then transmitted to a treatment member which is in direct contact with the body. The treatment member then broadcasts the selected neuro-electrical coded signals to an area in the body to control urinary function.

The neuro-electrical coded signals may be selected from a storage area in a computer, such as a scientific computer. The process of transmitting the selected neuro-electrical coded signals can either be done remotely or with the treatment member connected to a control module. The transmission may be seismic, electronic, photonic, magnetic, ultrasonic, or via any other suitable method.

The invention further provides an apparatus for controlling urinary function. The apparatus includes a source of collected neuro-electrical coded signals that are indicative of urinary function, a treatment member in direct contact with the body, means for transmitting collected waveforms to the treatment member, and means for broadcasting the collected neuro-electrical coded signals from the treatment member to the urinary bladder.

The transmitting means may include a digital to analog converter. The source of collected waveforms preferably comprises a computer which has the collected waveforms stored in digital format. The computer may include separate storage areas for collected neuro-electrical coded signals of different categories.

The treatment member may be comprised of an antenna or an electrode, or any other means of broadcasting one or more neuro-electrical coded signals directly to the body.

### BRIEF DESCRIPTION OF THE DRAWINGS

The invention is described in greater detail in the following description of examples embodying the best mode of the invention, taken in conjunction with the drawing figures, in which:

FIG. 1 is a schematic diagram of one form of apparatus for practicing the method according to the invention;

FIG. 2 is a schematic diagram of another form of apparatus for practicing the method according to the invention; and

FIG. 3 is a flow chart of the method according to the invention.

### DESCRIPTIONS OF EXAMPLES EMBODYING THE BEST MODE OF THE INVENTION

For the purpose of promoting an understanding of the principles of the invention, references will be made to the embodiments illustrated in the drawings. It will, nevertheless, be understood that no limitation of the scope of the invention is thereby intended, such alterations and further modifications in the illustrated device, and such further applications of the principles of the invention illustrated herein being contemplated as would normally occur to one skilled in the art to which the invention relates.

Skin usually has a 1,000 to 30,000 ohm resistance while the interior of the body is quite conductive. All neuro-electrical coded signals usually operate at less than 1 volt, naturally. Applied voltage may be up to about 20 volts according to the invention to allow for voltage loss during the transmission or conduction of the required coded signals through mylin nerve sheath or resistive fat and other material. Current should always be less than about 2 amps output for the invention. Direct conduction into the nerves via electrodes transmitting or connected directly to such nerves will likely have outputs of less than 3 volts and current of less than one-tenth of an amp. Up to 10 or more channels may be used simultaneously to exert medical treatment on glandular or muscular control to aid a patient in moving or performing muscular tasks suitable to his or her well-being as medical treatment with regard to ridding the body of urine.

Also claimed is the benefit of U.S. Patent Application entitled “Devices & Method for Conducting or Broadcasting Actual Neuro-Coded Signals for Medical Treatment” which holds Ser. No. 60/503,908 and was filed on Sep. 18, 2003. Such application teaches that electronic devices used to broadcast actual neuro-electrical coded signals into human or animal bodies to regulate individual organ functions. Such is incorporated herein by reference.

The invention encompasses both a device and a method for urinary function control by means of neuro-electrical coded signals. One form of a device 10 for urinary function control, as shown in FIG. 1, is comprised of at least one treatment member 12, and a control module 14. The treatment member 12 is in direct contact with a body and receives a neuro-electrical coded signal from the control module 14. The treatment member 12 may be an electrode, antenna, a seismic transducer, or any other suitable form of conduction attachment for broadcasting signals that regulate or operate urinary function in human or animals. The treatment member 12 may be attached to efferent nerves leading to the bladder, afferent nerves leading to the brain or brainstem to accomplish modulation of bladder output, the cervical spine, the neck, or the urinary bladder or urinary tract in a surgical process. Such surgery may be accomplished with “key-hole” entrance in a abdominal or pelvic stereo-scope procedure. If necessary a more expansive abdominal surgical or other approach may be required for more proper placement of the treatment member 12. Neuro-electrical coded signals known to modulate urinary function may then be sent into nerves that are in close proximity with the brain stem or other parts of the brain or close to the bladder itself.

The control module 14 is comprised of at least one control 16, and an antenna 18. The control 16 allows the device to regulate the signal transmission into the body. As shown in FIG. 1, the control module 14 and treatment member 12 can be entirely separate elements allowing the device 10 to be operated remotely. The control module 14 can be unique, or can be any appropriate conventional device which can provide neuro-electrical coded signals for transmission to the treatment member 12, which are similar to naturally occurring signals.

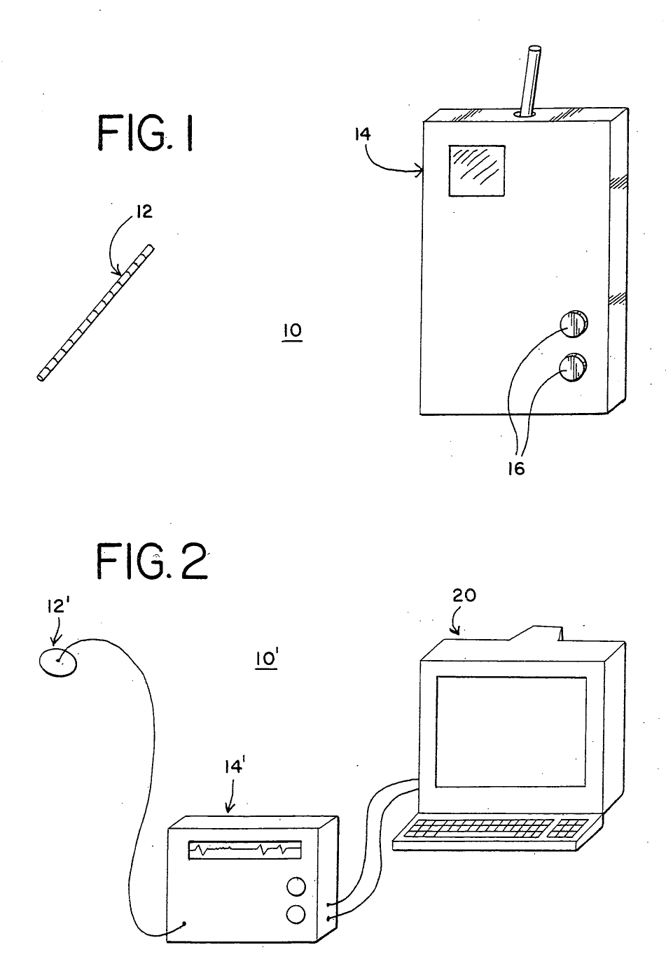

In an alternate embodiment of the device 10, as shown in FIG. 2, the control module 14′ and treatment member 12′ are connected. Similar members retain the same reference numerals in this figure. Additionally, FIG. 2 further shows another embodiment of the device 10′ as being connected to a computer 20, which provides greater capacity to store the neuro-electrical coded signals. The output voltage and amperage provided by the device 10′ during treatment shall not exceed 20 volts or 2 amps for each signal.

The computer 20 is used to store the unique neuro-electrical coded signals, which are complex and unique to urinary function. It is a neuro-electrical coded signal(s) selected from the stored library of neuro-electrical coded signals (waveforms) in the computer 20 which is transmitted to the control module 14′ and used for treatment of a patient. The neuro-electrical coded signals, and their creation, are described in greater detail in U.S. patent application Ser. No. 10/000,005, filed Nov. 20, 2001, and entitled “Device and Method to Record, Store, and Broadcast Specific Brain Waveforms to Modulate Body Organ Functioning,” the disclosure of which is incorporated herein by reference.

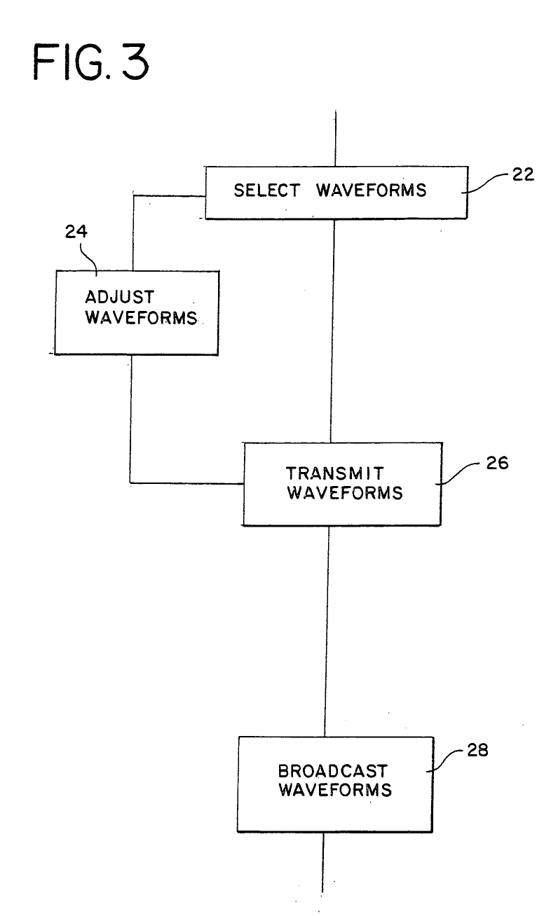

The invention further includes a method, as shown in FIG. 3, for using the device 10, 10′ for urinary function control. The method begins at step 22 by selecting one or more stored neuro-electrical coded signals from a menu of cataloged neuro-electrical coded signals. The neuro-electrical coded signals selected activate, deactivate, secrete, or adjust the urinary function(s). Such neuro-electrical coded signals are similar to those naturally produced by the brain structures for balancing and controlling urinary function. Once selected, the neuro-electrical coded signals may be adjusted, in step 24, to perform a particular urination related function in the body. Alternatively, if it is decided that the neuro-electrical coded signals do not need to be adjusted, step 24 is skipped and the process proceeds directly with step 26. At step 26, the neuro-electrical coded signal is transmitted to the treatment member 12, 12′ of the device 10, 10′.

Upon receipt of the neuro-electrical coded signals, the treatment member 12, 12′ broadcasts the neuro-electrical coded signals to the urinary bladder or nerve location, as shown in step 28. The device 10, 10′ utilizes appropriate neuro-electrical coded signals to adjust or modulate urinary action via conduction or broadcast of neuro-electrical coded signals into selected nerves. Controlling urinary function may require sending neuro-electrical coded signals into one or more nerves, including up to ten nerves simultaneously. It is believed that target nerves and organs can only “respond” to their own individual neuro-electrical coded signal. The operation of the urinary function requires that the selected neuro-electrical coded signals be silent and undetectable on certain selected nerves having to do with the evacuation of urine.

In one embodiment of the invention, the process of broadcasting by the treatment member 12, 12′ is accomplished by direct conduction or transmission through unbroken skin in a selected appropriate zone on the neck, head, spine, abdomen, or pelvis. Such zone will approximate a position close to the nerve or nerve plexus onto which the signal is to be imposed. The treatment member 12, 12′ is brought into contact with the skin in a selected target area that allows for the transport of the signal to the target nerve(s) via magnetic, photonic, ultrasonic or seismic methods.

In an alternate embodiment of the invention, the process of broadcasting the neuro-electrical coded signal is accomplished by direct conduction via attachment of an electrode to the receiving nerve or nerve plexus. This requires a surgical intervention as required to physically attach the electrode to the selected target nerve. Direct implantation on the nervous system of the urinary bladder may be performed in order to transmit signals to control all or some urinary function. Such implantation can be pre-synaptic or post-synaptic and may be attached to ganglion or nerve plexus -associated with the desired urinary function.

In yet another embodiment of the invention, the process of broadcasting is accomplished by transposing the neuro-electrical coded signal into a seismic form where it is sent into a region of the head, neck, spine, abdomen, or pelvis in a manner that allows the appropriate “nerve” to receive and to obey the coded instructions of such seismic signal. The treatment member 12, 12′ is pressed against the unbroken skin surface using an electrode conductive gel or paste medium to aid conductivity. The neuro-electrical coded signals may also be transmitted by light, sound, magnetic, ultrasonic, or seismic method depending on the injury or disease status of the patients urinary system.

Various features of the invention have been particularly shown and described in connection with the illustrated embodiments of the invention. However, it must be understood that these particular products, and their method of manufacture, do not limit but merely illustrate, and that the invention is to be given its fullest interpretation within the terms of the appended claims.

1. A method for controlling urinary function comprising the steps of:
a. selecting from a storage area one or more waveforms replicating those generated in the body and carried by neurons in the body;
b. transmitting or conducting the selected waveforms to a treatment member in contact with the body; and
c. broadcasting the selected waveforms from the treatment member to an area in the body is affected to control urinary function.

2. The method according to claim 1, in which step “a” further includes selecting said waveforms from a storage area in a computer.

3. The method according to claim 1, in which step “b” further comprises transmitting the selected waveforms remotely to the treatment member.

4. The method according to claim 1, in which step “b” further comprises seismic transmission of the selected waveforms.

5. The method according to claim 1, in which step “b” further comprises ultrasonic transmission of the selected waveforms.

6. The method according to claim 1, in which step “b” further comprises magnetic transmission of the selected waveforms.

7. An apparatus for controlling urinary function, comprising:
a. a source of collected waveforms generated in the body and indicative of urinary function;
b. a treatment member adapted to be in direct contact with the body;
c. means for transmitting one or more of the collected waveforms to the treatment member; and
d. means for broadcasting the collected waveforms from the treatment member to an area in the body such that urinary function is affected, thereby controlling urinary function.

8. The apparatus according to claim 7, in which said transmitting means includes a digital to analog converter.

9. The apparatus according to claim 7, in which said source comprises a computer having collected waveforms stored in digital format.

10. The apparatus according to claim 9, in which said computer includes separate storage areas for collecting waveforms of different urinary function categories.

11. The apparatus according to claim 7, in which the treatment member comprises an antenna for broadcasting urinary function signals.

12. The apparatus according to claim 7, in which the treatment member comprises an electrode.
